(19) United States
(12) Patent Application Publication
(10) Pub. No.: US 2004/0261798 A1
(43) Pub. Date: Dec. 30, 2004

(54) Nose filter

(76) Inventor: Ron Rimkus, Frankfort, IL (US)

Correspondence Address: Jeffery R. Gargano, Esq., Wallenstein Wagner & Rockey, Ltd., 53rd Floor, 311 South Wacker Drive, Chicago, IL, 60606-6630, US

(21) Appl. No.: 10/875,349

(22) Filed: Jun. 24, 2004

Related U.S. Application Data
(60) Provisional application No. 60/480,892, filed on Jun. 24, 2003.

(51) Int. Cl. A61M 16/00

(52) U.S. Cl. 128/206.11

(57) ABSTRACT

A disposable nose filter comprises a flexible housing, a positively ionized filtering component, a flange, an optional adhesive component, and an interconnecting component. The nose filter is adapted to be easily inserted and removed from the nostril. When positioned in the nasal passage of the nostril the nose filter becomes fixedly attached inside the nostril through the optional adhesive component, until removal. The interconnecting component prevents the nose filter from moving undesirably into the nasal cavity. The positively ionized filtering material attracts and retains negatively ionized particles from the air.

### RELATED APPLICATIONS

This Application is a nonprovisional of U.S. Provisional Patent Application No. 60/480,892, filed Jun. 24, 2003, and now pending. Priority to this provisional application is claimed under 35 USC § 119. The above-identified provisional application is also incorporated herein by reference.

### TECHNICAL FIELD

The present invention relates to disposable nose filters that, when positioned in a nasal passage of a nostril, become fixedly attached inside the nostril until removal due to an optional adhesive component on the outer surface of the nose filter. The filter portions of the nose filter are connected by a component external to the filter that protects against the filter moving further than necessary into the nostril and facilitates the removal of the filter from the nose. The filter portions of the nose filter are positively ionized to filter and purify air passing through the filter into the nostrils.

### BACKGROUND OF THE INVENTION

Disposable respiratory nose filters generally are of two types: mask-style nose filters, which are designed to cover the exterior of the nostrils of the nose, and intrusive-style filters, which are designed to be inserted into a nasal passageway of the nose.

U.S. Pat. No. 5,392,773 and the patents listed therein generally disclose mask-style nose filters. Generally speaking, mask-style nose filters are awkward, cumbersome, and unsightly. Further, when mask-like nose filters become moist, they can cling or stick to the face of the wearer, making speech and breath difficult.

Intrusive-style nose filters are disclosed in U.S. Pat. Nos. 3,457,917; 3,905,335; 4,052,983; 5,417,205; 5,568,808; and 5,890,491. U.S. Pat. No. 3,457,917 discloses a nondisposable nose filter that includes a filter retaining means (i.e. a housing), filtering material, and a detachable cap having a finger gripping means provided at the end of the cap. Unlike the present invention, the filter retaining means in U.S. Pat. No. 3,457,917 is formed from a hard molded plastic such as polytetrafluorethylene or Bakelite.

U.S. Pat. No. 3,905,335 discloses a nasal air filter comprising a pair of interconnected cylinders having a filter material contained within. The cylinders further contain yieldable flange means which engage the inner nostril walls.

U.S. Pat. No. 4,052,983 discloses a filter device insertable into the nasal passages that includes a pair of flexible casings, each casing releasably receiving a filter cartridge. The filter cartridges have a multitude of cilia-like nylon filaments that act to electrostatically charge the air as it moves past the filaments, thus aiding in the filtering process.

U.S. Pat. No. 5,417,205 discloses an air filter comprised of two filter units linked by a connecting element. Each filter unit further comprises first and second gauze filters and a plurality of rods that extend from the first gauze filter to the second gauze filter and retain a stack of wet filter cloth between the gauze filters.

U.S. Pat. No. 5,568,808 discloses an air filter generally comprised of a housing, a filter component, and a flutter valve. Upon inhalation the flutter valve forms a seal with the lower external portion of the nostril, forcing the air to pass through the filter component. Upon exhalation the seal between the flutter valve and the external portion of the nostril is broken, allowing air to escape through the area between the housing and the inner wall of the nostril. If such an air filter is used with an over-abundance of medicant or aqueous solutions, the broken seal would allow the medicant or solutions to escape from the nostril.

U.S. Pat. No. 5,890,491 discloses an air filter that includes a flexible housing, a flange, a filtering component, and an adhesive component. When positioned in the nasal passage of a nostril, the filter becomes fixedly attached inside the nostril through the adhesive component. The flange forms a seal with the lower exterior portion of the nostril, thus forcing air through the filter and preventing air from passing between the housing and inner walls of the nostril.

An improved nose filter in accordance with the present invention eliminates the disadvantages and unpleasantries of the prior nose filters described above.

### SUMMARY OF THE INVENTION

The nose filter of the present invention is adapted to be inserted into the nostrils and includes a flexible housing, a filtering component, a flange, an optional adhesive component, and an interconnecting component (i.e., a safety strap) connecting the housing components. The housing includes an open bottom end, a top end, and a side wall having a top portion and a bottom portion. A cavity is disposed within the housing and being inside the side wall and between the top end and the bottom end of the housing. The filtering component is disposed within the cavity of the housing. The flange has a diameter larger than the diameter of the inner wall of the nostril and extends radially outwardly from the wide wall of the housing. Optionally, an adhesive component is applied to an outer surface of the flexible housing or, alternatively, to a first surface of the flange, or to both the housing and the flange. The interconnecting component is connected to the surface of the flange or, alternatively, to the surface of the housing, thus connecting multiple nose filters together. The filtering component is mildly positively ionized.

The connecting safety strap between the two nasal filter housings prevents the filter from being forced too far into the nasal cavity or into the sinus area, where the filter may be drawn into the lung area causing infection and possibly leading to surgery for extraction. The safety strap can be attached to the unique flange/valve, which when placed into the nasal opening is on the outside of the nose area thus allowing a completely air-tight fit and continuing to disallow unfiltered air to pass between the housing and the inner wall of the nostril. Straps on other nasal filter devices do not have the unique exterior flange/valve and must be attached to the side of the housing and inserted into the nostril not allowing for an air-tight fit because they attach to the side wall of the device and not to a unique exterior of the nose flange.

Upon insertion of the nose filter into the nostrils, the first surface of the flange engages and forms a seal with either an external lower portion of the nostrils or a portion of the inner wall of the nostrils. Additionally, upon insertion, the optional adhesive component of the nose filter adhesively attaches the nose filter within the nasal passage of the nostrils until removal.

According to a first aspect of the present invention, disposable interconnected nose filters filter dust, germs, allergens, and other foreign particulates from air passing through the nostrils into the sinus cavity. The positive ionic charge of the filtering component attracts and retains the negatively-ionized particulate matter from air passing through the nostrils into the sinus cavity. It has been found that as air passes through the nose filter, air is also warmed before it reaches the sinus cavity and eventually the lungs.

In another aspect of the present invention, disposable interconnected nose filters form seals with the nostrils, thus forcing air through the filter and preventing air from passing between the nose filter and the inner walls of the nostril. Additionally, the filter in each nostril is interconnected to the filter in the other nostril, thereby fixing the nose filter in place such that both filters are quickly and easily removable from the nose. The interconnecting component between the filters further prevents the filter in each nostril from sliding further than desired into the nasal cavity.

In another aspect of the present invention, a prefiltering component is located proximate to the bottom portion of the filtering component. The prefiltering component filters out large particles from entering the nasal cavity, thus preventing such particles from entering the filtering component of the nose filter. This allows the filtering component to be more efficient in purifying and warming inhaled air and increases the longevity of the filtering component.

In yet another aspect of the present invention, a nose filter moistens the mucous membranes of the sinus cavity and prevents dryness or irritation due to pollen, smoke, industrial chemicals, perfumes, dust, mold, and other allergens.

In a final aspect of the present invention, a nose filter can be easily inserted and removed from the nostril and that conforms to the shape of nasal passageway.

Other advantages and aspects of the present invention will become apparent upon reading the following description of the drawings and detailed description of the invention.

While this invention is susceptible of embodiment in many different forms, there is shown in the drawings and herein described in detail preferred embodiments with the understanding that the present disclosure is to be considered an exemplification of the principles of the invention and is not intended to limit the broad aspects of the invention to the embodiments illustrated.

### BRIEF DESCRIPTION OF THE DRAWINGS

FIG. 1 is an angled view of the nose filter according to the present invention wherein the filters are interconnected by the interconnecting component attached to each filter; and

FIG. 2 is a front view of a nose filter according to the present invention inserted into the nostrils wherein the interconnecting portion of the filter extends underneath the nose and across the philtral columns of the upper lip.

### DETAILED DESCRIPTION OF THE DRAWINGS

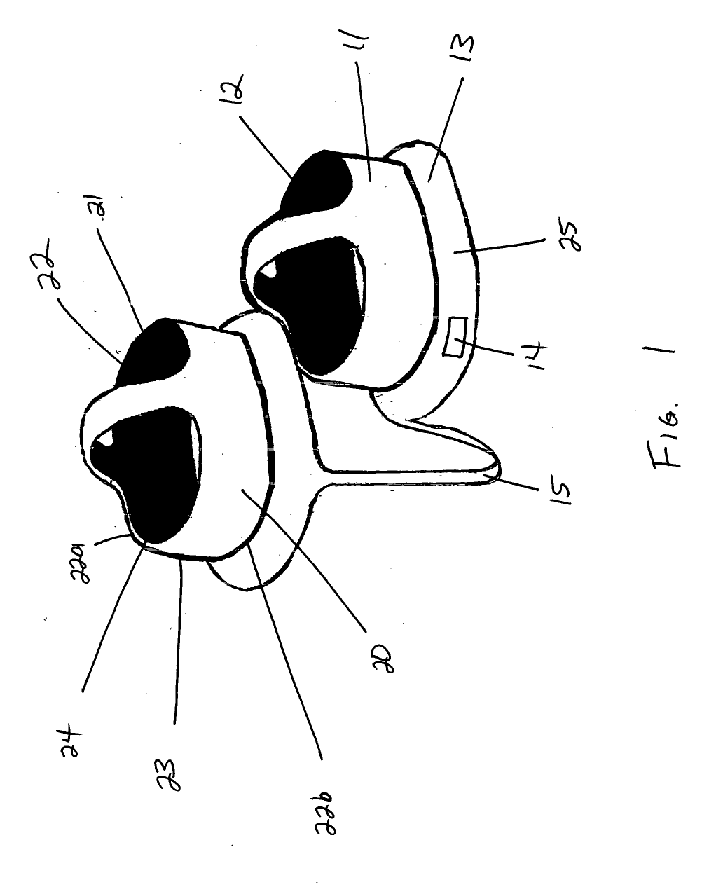

Referring in detail to the drawings and initially to FIG. 1, a preferred embodiment of nose filter 10 is shown constructed in accordance with the present invention. The nose filter 10 preferably comprises a flexible housing 11, a filtering component 12, a flange 13, an optional adhesive component 14, and an interconnecting component 15. The flexible housing 11 has an open bottom end 20, a top end 21 having at least one passageway 22, a side wall 23 having a top portion 22a and a bottom portion 22b, and a cavity 24 located within the housing 11 and being inside the side wall 23 and the bottom end 20 of the housing 11. The top end 21 of the flexible housing 11 has a plurality of passageways 22. Also, the filtering component 12 has a top portion 12a and a bottom portion 12b and is disposed within the housing cavity 24.

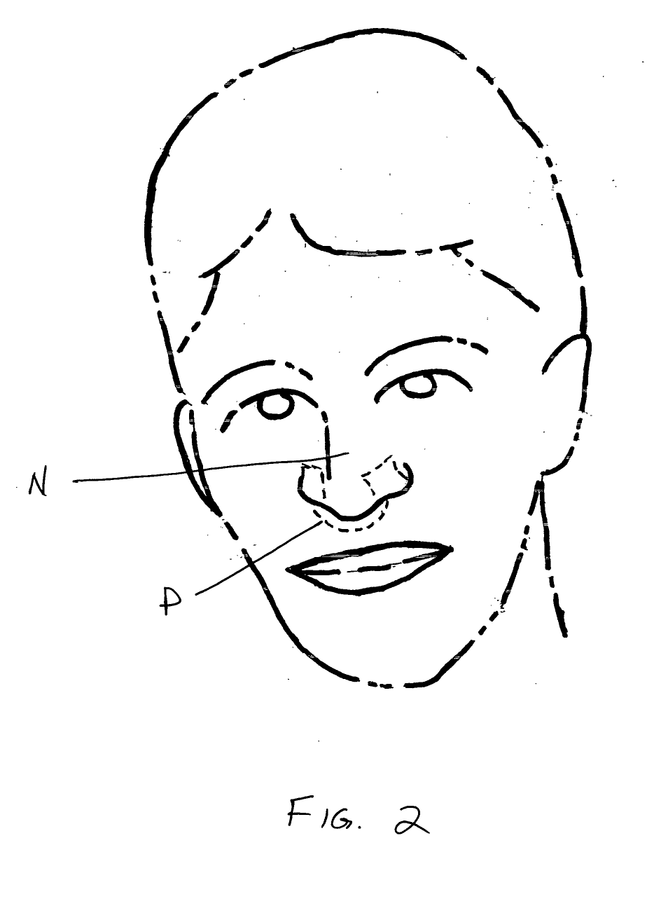

In the embodiment illustrated in FIG. 2, the nose filter is inserted into the nostrils of the nose N. As shown in FIG. 2, the flange 13 has a diameter larger than the diameter of the inner wall of the nostril N and can be connected to the nose filter 10 at either the bottom portion 22b of the side wall 22 or to the bottom portion 12b of the filtering component 12. In either embodiment, the flange 13 extends radially outwardly from the bottom portion 22b of the side wall 22 so that when the nose filter 10 is inserted into the nostril N, the flange 13 is partially exposed from the nostril N and engages the lower portion P of the nostril N. However, the flange 13 may also engage the internal wall of the nostril N if the nose filter 10 is inserted high enough into the nasal passage of the nostril N.

The flange 13 further has a first surface 25. With reference to FIG. 1, the interconnecting component 15 may be connected to the first surface 25 of the flange 13. Alternatively, the interconnecting component 15 may be connected to the side wall 23 or bottom end 20 of the housing 11. The filtering component 12 contains positively ionized filtering material.

Therefore, as the person wearing the nose filter 10 breathes, the flange 13 and the entire nose filter 10 remains stationary. The interconnecting component 15 prevents the flexible housing 11 or any other portion of the nose filter 10 from moving undesirably into the nostril N. The person wearing the nose filter 10 may quickly and easily remove the nose filter 10 from the nostrils N by pulling on the interconnecting component 15. As a result, the nose filter 10 of the present invention remains removeably secure within the nostrils N while being safely prevented from entering undesirably further into the nostrils N.

Since the filtering component 12 of the nose filter 10 contains positively ionized filtering material, the positively ionized material attracts the negatively ionized particulate matter in the air. Since the smallest particulate matter in the air may not be removed from the air with the use of conventional filtering material, the positively ionized filtering material in the filtering component 12 of the present invention attracts these negatively ionized particles and prevents them from entering the nostrils N.

In the embodiments of the present invention illustrated in FIGS. 1 and 2, any suitable plastic or rubber-like material, including a synthetic rubber latex or silicone, which is nontoxic and which does not irritate the inner wall of the nostril N, may be used to form flexible housing 11. In a preferred embodiment, flexible housing 11 is formed with a silicone rubber material. However, the present invention also contemplates embodiments where flexible housing 11 comprises a naturally porous material such as cotton, linen, or gauze. It has been found that when flexible housing 11 is formed of a plastic or rubber-like material and the nose filter 10 is interested into the nostril N, flexible housing 11 also aids in forcing the nasal passageway open, thus allowing more air to pass through the nose filter 10 and into the sinus cavity.

When the nose filter 10 of the present invention is not in use, it may be kept in a convenient container with a saline or other aqueous solution to keep the filtering component moist. It will also be understood that the size and shape of the nose filter 10 of the present invention may be varied to accommodate noses of different shapes and sizes.

While the specific embodiments have been illustrated and described, numerous modifications come to mind without markedly departing from the spirit of the present invention. The scope of protection is only intended to be limited by the scope of the accompanying claims.

What I claim is:

1. A nose filter for insertion into a nostril, the nostril including an inner wall having a diameter and an external lower portion, the nose filter comprising:
a flexible housing having an open bottom end, a top end, a side wall having a top portion and a bottom portion, and a cavity located inside the side wall and between the top end and the bottom end of the housing;
a filtering component disposed in the housing cavity, the filtering component having a top portion and a bottom portion, wherein the filtering component is positively ionized;
a flange having a diameter larger than the diameter of the inner wall of the nostril, the flange extending radially outwardly from the bottom end of the flexible housing and having a first surface which forms a seal with the nostril; and,
an interconnecting component connected to either the housing or the flange wherein the interconnecting component connects one nose filter to another nose filter.

2. The nose filter of claim 1, wherein the flange is connected to the bottom portion of the filtering component.

3. The nose filter of claim 1, wherein the flange is connected to the bottom portion of the side wall of the flexible housing.

4. The nose filter of claim 1, further comprising an adhesive component applied to the nose filter.

5. The nose filter of claim 4, wherein the adhesive component comprises an adhesive disposed about at least a portion of a circumference of the flexible housing.

6. The nose filter of claim 4, wherein the adhesive component comprises an adhesive disposed about at least a portion of the first surface of the flange.

7. The nose filter of claim 4, wherein the adhesive component comprises an adhesive disposed about at least a portion of both the first surface of the flange and a circumference of the flexible housing.

8. The nose filter of claim 1, wherein the first surface of the flange forms a seal with the external lower portion of the nostril.

9. The nose filter of claim 1, wherein the first surface of the flange forms a seal with the inner wall of the nostril.

10. The nose filter of claim 1, wherein the flexible housing is formed from a silicone rubber material.

11. The nose filter of claim 1, wherein the thickness of the flange is less than half the thickness of the side wall of the flexible housing.

12. The nose filter of claim 1, wherein the thickness of the flange is approximately the same as the thickness of the side wall of the flexible housing.

13. The nose filter of claim 1, wherein the flexible housing comprises a natural porous material selected from the group consisting of cotton, linen, and gauze.

14. The nose filter of claim 1, wherein the filtering component comprises an absorbent material.

15. The nose filter of claim 14, wherein the absorbent material is soaked in an aqueous solution.

16. The nose filter of claim 1, further comprising a pre-filtering component proximate the bottom portion of the filtering component.

17. The nose filter of claim 1, wherein the thickness of the interconnecting component is less than half the thickness of the side wall of the flexible housing.

18. The nose filter of claim 1, wherein the thickness of the interconnecting component is approximately the same as the thickness of the side wall of the flexible housing.

19. A nose filter for insertion into a nostril, the nostril including an inner wall having a diameter and an external lower portion, the nose filter comprising:
a filtering component having a top portion and a bottom portion, wherein the filtering component is positively ionized;
a flange having a diameter larger than the diameter of the inner wall of the nostril, the flange extending radially outwardly from the filtering component and having a first surface which forms a seal with the nostril; and,
an interconnecting component connected to either the housing or the flange wherein the interconnecting component connects one nose filter to another nose filter.

20. The nose filter of claim 19, further comprising an adhesive component applied to the nose filter.

21. The nose filter of claim 20, wherein the adhesive component comprises an adhesive disposed about at least a portion of a circumference of the filtering component.

22. The nose filter of claim 20, wherein the adhesive component comprises an adhesive disposed about at least a portion of the first surface of the flange.

23. The nose filter of claim 20, wherein the adhesive component comprises an adhesive disposed about at least a portion of both the first surface of the flange and a circumference of the filtering component.

24. The nose filter of claim 19, wherein the first surface of the flange forms a seal with the external lower portion of the nostril.

25. The nose filter of claim 19, wherein the first surface of the flange forms a seal with the inner wall of the nostril.

26. The nose filter of claim 19, wherein the flange is formed of a silicone rubber material.

27. The nose filter of claim 19, wherein the filtering component comprises an absorbent material.

28. The nose filter of claim 19, further comprising a pre-filtering component proximate the bottom portion of the filtering component.

29. A nose filter for insertion into a nostril, the nostril including an inner wall having a diameter and an external lower portion, the nose filter comprising:
a flexible housing having an open bottom end, a top end, a side wall having a top portion and a bottom portion, and a cavity located inside the side wall between the top end and the bottom end of the housing;
a filtering component disposed in the housing cavity, the filtering component having a top portion and a bottom portion, wherein the filtering component is positively ionized;
a flange having a diameter larger than the diameter of the inner wall of the nostril, the flange extending radially outwardly from the bottom end of the flexible housing and having a first surface which forms a seal with the nostril;
a pre-filtering component proximate the bottom portion of the filtering component; and,
an interconnecting component connected to either the housing or the flange wherein the interconnecting component connects one nose filter to another nose filter.
